(19) United States
(12) Patent Application Publication
(10) Pub. No.: US 2013/0210014 A1
(43) Pub. Date: Aug. 15, 2013

(54) METHOD FOR DETERMINING THE PROGNOSIS AND THERAPEUTIC RESPONSE IN CHRONIC LYMPHOCYTIC LEUKEMIA (CLL) PATIENTS

(71) Applicant: Jeff Sharman, Eugene, OR (US)

(72) Inventor: Jeff Sharman, Eugene, OR (US)

(21) Appl. No.: 13/761,957

(22) Filed: Feb. 7, 2013

Related U.S. Application Data
(60) Provisional application No. 61/597,594, filed on Feb. 10, 2012.

(51) Int. Cl. C12Q 1/68

(52) U.S. Cl. CPC C12Q 1/68
USPC 435/6.12

(57) ABSTRACT

The present invention is based on the seminal discovery that using next generation sequencing, PCR or the like, one of skill in the art can determine the prognosis for or predict responsiveness to a particular therapeutic regimen in a subject diagnosed with CLL.

### CROSS-REFERENCE TO RELATED APPLICATION

This application claims benefit of priority under 35 U.S.C. §119(e) of U.S. Ser. No. 61/597,594, filed Feb. 10, 2012, the entire contents of which are incorporated herein by reference in its entirety.

### BACKGROUND INFORMATION

1. Field of the Invention

The present invention relates generally to diagnostic and prognostic methods and more specifically to prognosis and predicting responsiveness in CLL patients.

2. Background of the Invention

Chronic Lymphocytic Leukemia (CLL) is the most common adult leukemia. Although it has an annual incidence of approximately 15,000 cases per year in the United States, it is frequently associated with an indolent clinical course and the prevalence is over 100,000 patients alive with the disease.

CLL is a model disease for research because of the ease of access to malignant cells. As a result, considerable insight has been gained into the biology of this disease. Many easily measurable DNA markers confer both prognostic and predictive insight. In contrast to CML in which most clinically relevant tests can be performed on a single gene target BCR-Abl, CLL has considerably more markers that are clinically significant. Relevant examples used today include the deletion of chromosome 11q (ATM protein) indicates the need for alkalator based therapy, or loss of chromosome 17p (p53 protein) for which early intensive approaches including transplant are appropriate.

With the introduction of next generation sequencing technologies, numerous novel mutations have been discovered. In many cases, these mutations have been characterized in the context of clinical data sets and are therefore also important for both prognosis and prediction. Examples include recently discovered mutations in NOTCH which confer equally poor outcome as p53 loss. Others include BIRC3 or SF3B1 which predict fludarabine or other purine analog resistance. In addition, an increasing number of investigational drugs are linked to individual mutations and could help select patients for clinical trials or particular therapies or studies.

Several problems exist with currently clinically available clinically diagnostic tests. The most frequently utilized test FISH only looks for large scale copy number alterations. In the case of deletion 13p the size of the deletion matters as those that include the Rb protein confer a worse prognosis than those that retain expression of RB. Furthermore, point mutations are completely ignored by this technology, yet mutation of p53 or ATM confers prognosis similar to deletion. Perhaps the most important deficiency in current FISH testing is misinterpretation or misunderstanding of the results on by practicing clinicians who are not familiar with the significance of the results.

Virtually all CLL patients seen in tertiary centers and many patients in community practice are evaluated for mutation status of BCR and cytogenetics via FISH. As the disease evolves, both cytogenetics and mutations change. It is therefore fairly common to repeat FISH prior to any new line of therapy.

There is a significant clinical need for a comprehensive molecular profile for patients with CLL that provides user friendly clinical interpretation of testing results. Target genes and mutations have largely already been identified through genomic sequencing efforts and those targets are rapidly becoming clinically annotated.

### SUMMARY OF THE INVENTION

In one embodiment, the invention provides a method for determining the prognosis for or predicting responsiveness to a therapeutic regimen for a subject with chronic lymphocytic leukemia (CLL) including determining the copy number in chromosome 17p and/or 11q or the presence of mutations in p53 and/or ATM in a sample from the subject, wherein a correlation between copy number alterations and the presence of mutations is indicative of a subject's prognosis or likelihood of responsiveness to a therapeutic regimen.

The method of the invention may further include detecting copy number alterations in chromosome 11q, trisomy12, 13q and/or 17p. In one aspect, the method may further include detecting mutations in one or more of p53, ATM, SF3B1, MYD88, FBXW7, NOTCH1, BIRC3, IRF4 and any combination thereof.

In one aspect, there is a deletion in 13q containing all or a portion of the RB gene. In one aspect, there is a deletion in 11q containing all or a portion of BIRC3.

In one aspect, the distribution of mutations and wild type sequences determines the clonal frequency using next generation sequencing or PCR.

In one embodiment, malignant conditions with elevated WBCs are further evaluated be analyzing mutations in BRAF or NOTCH2, or translocations in chromosomes 11;14 or 14;18.

In one aspect, alterations of ATM or 11q predict need for alkylator based therapy. In another aspect, alterations of p53, 17p, SF3B1, BIRC3 predict lack of benefit from fludarabine based therapy.

In yet another aspect, an adverse prognosis is conferred by alterations in p53, 17p, Notch, SF3B1, BIRC3 or a combination thereof.

The method of the invention includes a sample such as a blood sample, lymph node or bone marrow aspirate. Any B cell containing biological sample is envisioned in the method of the invention. While not necessary, in one aspect B cells are isolated from the sample.

In another embodiment, the invention provides a kit for determining the prognosis for or predicting responsiveness to a therapeutic regimen for a subject with chronic lymphocytic leukemia (CLL). The kit includes primers specific for amplification of chromosome 11q, 17p or mutations in p53 and ATM. In one aspect, the amplification is performed in a next generation sequencing multiplex format.

In one aspect, the kit further includes primers for amplification of at least one of chromosome 13q, trisomy 12, or mutations in SF3B1, MYD88, FBXW7, NOTCH1, BIRC3, IRF4, and any combination thereof.

In yet another embodiment, the invention provides a method for determining prognosis for a subject having CLL comprising determining the extent of copy number alterations of chromosome 13q, wherein a deletion including the RB gene is indicative of a poor prognosis as compared with a subject not having a deletion including the RB gene.

### DETAILED DESCRIPTION OF THE INVENTION

The present invention is based on a prognostic test for CLL that predicts clinical responsiveness in a patient. Ideally, the invention test would replace outdated FISH technology. It should capture focused copy number alterations of chromosomes 11q, 12, 13p, 13q, and 17p as well as mutations in key CLL target proteins including, p53, SF3B1, MYD88, ATM, FBXW7, NOTCH1, BIRC3, XPO, and others. Since there are a variety of conditions that may clinically resemble CLL that are known to have unique molecular alterations, these would be evaluated as well in the event that a patient was misdiagnosed (t14:18 for follicular lymphoma, B-RAF V600E mutation for hairy cell leukemia, t11:14 for mantle cell lymphoma, NOTCH2 for marginal zone lymphoma and t14:18 for follicular lymphoma).

The present invention is based on the seminal discovery that using next generation sequencing, PCR or the like, one of skill in the art can determine the prognosis for or predict responsiveness to a particular therapeutic regimen in a subject diagnosed with CLL. In one embodiment, the invention provides a method for determining the prognosis for or predicting responsiveness to a therapeutic regimen for a subject with chronic lymphocytic leukemia (CLL) which includes: determining the copy number in chromosome 17p and/or 11q or the presence of mutations in p53 and/or ATM in a sample from the subject, wherein a correlation between copy number alterations and the presence of mutations is indicative of a subject's prognosis or likelihood of responsiveness to a therapeutic regimen. The method may further include detecting copy number alterations is chromosome 11q, trisomy12, 13p, 13q, and/or 17p. In one aspect, the invention method further includes detecting mutations in one or more of SF3B1, MYD88, FBXW7, NOTCH1, BIRC3, IRF4 and any combination thereof.

The invention assay includes peripheral blood subjected to B cell isolation. Malignant cells would then undergo sequencing (e.g., next generation, PCR and the like) of targeted genes and copy number alterations with sufficient depth of coverage to detect small clones of disease within a larger population of malignant cells. A report would be generated which characterizes the significance of findings unique to the sample such as chemosensitivity, prognosis, familial inheritance, etc.).

Ideally, the invention test would be utilized in manner similar to OncotypeDX in breast and colon cancer (Genomic Health) or the Canis Diagnostic test (Canis) except it would be applied exclusively to CLL. By way of example, the Oncotype DX test is a genomic test that analyzes the activity of a group of genes that can affect how a cancer is likely to behave and respond to treatment. The Oncotype DX is used in two ways: to help doctors figure out a woman's risk of early-stage, estrogen-receptor-positive breast cancer coming back (recurrence), as well as how likely she is to benefit from chemotherapy after breast cancer surgery and; to help doctors figure out a woman's risk of DCIS (ductal carcinoma in situ) coming back (recurrence) and/or the risk of a new invasive cancer developing in the same breast, as well as how likely she is to benefit from radiation therapy after DCIS surgery. The results of the Oncotype DX test, combined with other features of the cancer, can help you make a more informed decision about whether or not to have chemotherapy to treat early-stage hormone-receptor-positive breast cancer or radiation therapy to treat DCIS. In a similar manner, the present invention CLL test will be used to assist the physician and the patient in the therapeutic regimen chosen for that patient.

The present invention is based on the seminal discovery that using next generation sequencing, PCR or the like, one of skill in the art can determine the prognosis for or predict responsiveness to a particular therapeutic regimen in a subject diagnosed with CLL. In one embodiment, the invention provides a method for determining the prognosis for or predicting responsiveness to a therapeutic regimen for a subject with chronic lymphocytic leukemia (CLL) which includes: determining the copy number in chromosome 17p and/or 11q or the presence of mutations in p53 and/or ATM in a sample from the subject, wherein a correlation between copy number alterations and the presence of mutations is indicative of a subject's prognosis or likelihood of responsiveness to a therapeutic regimen. The method may further include detecting copy number alterations is chromosome 11q, trisomy12, 13q, and/or 17p. In one aspect, the invention method further includes detecting mutations in one or more of SF3B1, MYD88, FBXW7, NOTCH1, BIRC3, IRF4 and any combination thereof.

Detection of a deletion in 13q containing all or a portion of the RB gene provides further information as to prognosis. The distribution of mutations and wild type sequences determines the clonal frequency using next generation sequencing. Malignant conditions with elevated WBC can be further evaluated be analyzing mutations in BRAF, or translocations in chromosomes 11;14 or 14;18, for example.

The term “treatment” as used herein includes partial or total destruction of the lymphoid malignancies with minimal destructive effects on normal cells.

The terms “therapeutically effective” and “pharmacologically effective” are intended to qualify the amount of each agent which will achieve the goal of improvement in disease severity and the frequency of incidence, while avoiding adverse side effects typically associated with alternative therapies. Examples of drugs selected for treatment of a patient with CLL, depending on age, stage of CLL, genetic disposition, and the like, include but are not limited to:
FCR: fludarabine (Fludara), cyclophosphamide (Cytoxan), and rituximab (Rituxan)
PCR: pentostatin (Nipent), cyclophosphamide, and rituximab
FR: fludarabine and rituximab
CVP: cyclophosphamide, vincristine, and prednisone (sometimes with rituximab)
CHOP: cyclophosphamide, doxorubicin, vincristine (Oncovin), and prednisone
Chlorambucil and prednisone
Bendamustine (sometimes with rituximab)
Alemtuzumab (Campath)
Fludarabine (sometimes with rituximab)

Other drugs or combinations of drugs may also be also used.

The term “subject” for purposes of treatment includes any human or animal subject who has a disorder characterized by lymphoid malignancies or is at risk of developing such a disorder. Such disorders include, but are not limited to chronic lymphocytic leukemia (CLL). For methods described herein, the subject is any human or animal subject, and in some embodiments, the subject is a human subject who has developed or is at risk of developing a lymphoid malignancy. The subject may be at risk due to exposure to carcinogenic agents, being genetically predisposed lymphoid malignancies, and so on. Besides being useful for human treatment, the compounds of the present invention are also useful for veterinary treatment of mammals, including companion animals and farm animals, such as, but not limited to dogs, cats, horses, cows, sheep, and pigs.

“CLL”, as used herein, refers to chronic lymphocytic leukemia involving any lymphocyte, including but not limited to various developmental stages of B cells and T cells, including but not limited to B cell CLL. B-CLL, as used herein, refers to leukemia with a mature B cell phenotype which is CD5+, CD23+, CD20+, sIg+.

The lymphocytes analyzed in the method of the present invention can be obtained by any method. For example, the lymphocytes can be present in blood collected using standard phlebotomy techniques. Bone marrow comprising lymphocytes can be obtained by aspiration or biopsy using a needle inserted into the bone.

Nucleic acids in the lymphocytes can be assayed by a variety of methods for detecting chromosomal deletions, such as northern blots, Southern blots, restriction fragment length polymorphism analysis, the polymerase chain reaction (PCR), next generation sequencing, reverse transcriptase PCR (RT-PCR), rapid amplification of cDNA ends (RACE), FISH assays, or any method known to those skilled in the art for nucleic acid detection. Preferably, next generation sequencing is used.

In one embodiment, the invention provides a method determining prognosis for a subject having CLL including determining the extent of copy number alterations of chromosome 13q, wherein a deletion including the retinoblastoma (RB) gene is indicative of a poor prognosis as compared with a subject not having a deletion including the RB gene.

In one aspect, alterations of ATM or 11q predict need for alkylator based therapy, and alterations of p53, 17p, SF3B1, BIRC3 predict lack of benefit from fludarabine based therapy. Further, in the method of the invention, an adverse prognosis is conferred by alterations in p53, 17p, Notch, SF3B1, BIRC3 or a combination thereof.

The sample can be any biological sample, including a blood sample, lymph node or bone marrow aspirate by way of example. Further, B cells are isolated from the sample.

In another embodiment, the invention provides a kit for determining the prognosis for or predicting responsiveness to a therapeutic regimen for a subject with chronic lymphocytic leukemia (CLL) including: primers specific for amplification of chromosome 11q, 17p or mutations in p53 and ATM. Preferably, the amplification is performed in a next generation sequencing multiplex format.

In one aspect, the kit further includes primers for amplification of at least one of chromosome 13q, trisomy 12, or mutations in SF3B1, MYD88, FBXW7, NOTCH1, BIRC3, IRF4, and any combination thereof. Further, the invention methods and kits may include primers and/or probes for the B cell receptor (BCR) to identify it as “mutated” or “unmutated”. Unlike chronic myeloid leukemia, chronic lymphocytic leukemia (CLL) lacks a common genetic aberration amenable to therapeutic targeting.

Recent research has revealed the existence of subsets (clusters) of patients with different types of B-cell lymphomas and leukemias with restricted, “stereotyped” immunoglobulin (IG) variable heavy complementarity-determining region 3 (VH CDR3) sequences within their B cell receptors (BcR), suggesting selection by common epitopes or classes of structurally similar epitopes. BcR stereotypy was initially described in chronic lymphocytic leukemia (CLL), where it constitutes a remarkably frequent feature of the IG repertoire, and subsequently identified in other malignancies, including mantle cell lymphoma and splenic marginal-zone lymphoma. The criteria for identification of BCR stereotypy and its actual frequency as well as the identification of “CLL-biased” features in BCR Ig stereotypes have been investigated. CLL may be subdivided into 2 distinct categories: one with stereotyped and the other with non-stereotyped BCRs, at an approximate ratio of 1:2, and evidence suggests a different ontogeny for these 2 categories. This compartmentalized examination of VH sequences may pave the way toward a molecular classification of CLL with implications for targeted therapeutic interventions, applicable to a significant number of patients assigned to the same subset. Of note, at least in CLL, emerging evidence indicates that the grouping of cases into distinct clusters with stereotyped BcR is functionally and prognostically relevant. Hence, the reliable identification of BcR stereotypy may assist in the investigation of the nature of the selecting antigens and immune pathways leading to lymphoma development, and also potentially pave the way for tailored treatment strategies applicable to each major stereotyped subset (Darzentas et al., Methods in Molecular Biology, Vol. 971, Apr. 1, 2013, pp. 135-148).

Kits of the invention comprise reagents suitable for detecting, measuring, sequencing, or otherwise analyzing, for example, chromosome 17p and/or 11q or the presence of mutations in p53 and/or ATM in a sample from the subject. In some embodiments, the kit includes a reagent for evaluating the chromosome locations (e.g., primers and/or probes for evaluating expression or sequence) in a sample; and (b) reagents for evaluating the status of p53 and/or ATM, for example, (e.g., primers and/or probes for evaluating expression or sequence) in a sample. It is contemplated that the reagents for evaluating the level of expression or activity of any of p53 and/or ATM can be one or more nucleic acids. The nucleic acids may be complementary to all or part of the gene or its product and they can be used in hybridization reactions, such as for amplification (primers), primer extensions, nuclease protection assays, Northern blotting, or with an array or other structure. Alternatively, antibodies against the proteins can be used, for example, in Western blotting, ELISAs, other sandwich assays, antibody arrays, IHC, or FACS analysis. The antibody may be a monoclonal or a polyclonal antibody. The kits of the invention may further comprise additional reagents suitable for performing hybridization and/or amplification reactions. The kit may further include reagents for detecting copy number alterations is chromosome 11q, trisomy12, 13p, 13q, and/or 17p. In one aspect, the invention kit further includes reagents for detecting mutations in one or more of SF3B1, MYD88, FBXW7, NOTCH1, BIRC3, IRF4 and any combination thereof.

The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit may also include instructions on the interpretation of the results of the test performed, e.g., instructions explaining what the results may mean and optionally indicates normal or increased likelihood of response to a particular therapy.

Reports, Programmed Computers, Business Methods, and Systems

The results of a test (e.g., an individual's prognosis or an individual's predicted drug responsiveness), based on an invention method as disclosed herein, and/or any other information pertaining to a test, may be referred to herein as a “report”. A tangible report can optionally be generated as part of a testing process (which may be interchangeably referred to herein as “reporting”, or as “providing” a report, “producing” a report, or “generating” a report).

Examples of tangible reports may include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which may optionally be accessible via the internet (such as a database of patient records or genetic information stored on a computer network server, which may be a “secure database” that has security features that limit access to the report, such as to allow only the patient and the patient's medical practitioners to view the report while preventing other unauthorized individuals from viewing the report, for example). In addition to, or as an alternative to, generating a tangible report, reports can also be displayed on a computer screen (or the display of another electronic device or instrument).

A report can include, for example, an individual's prognosis or likelihood of responding to a particular therapeutic regimen for CLL, or may just include the allele(s)/genotype that an individual carries at one or more chromosomal locations or genes as disclosed herein, which may optionally be linked to information regarding the significance of having the particular allele(s)/genotype (e.g., status of the BCR) (for example, a report on computer readable medium such as a network server may include hyperlink(s) to one or more journal publications or websites that describe the medical/biological implications, such as increased or decreased drug responsiveness, for individuals having a certain allele/genotype at the location of interest). Thus, for example, the report can include disease risk or other medical/biological significance (e.g., drug responsiveness, etc.) as well as optionally also including the allele/genotype information, or the report may just include allele/genotype information without including disease risk or other medical/biological significance (such that an individual viewing the report can use the allele/genotype information to determine the associated disease risk or other medical/biological significance from a source outside of the report itself, such as from a medical practitioner, publication, website, etc., which may optionally be linked to the report such as by a hyperlink).

A report can further be “transmitted” or “communicated” (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the interne, by facsimile, and/or by any wired or wireless communication methods known in the art) such as by being retrieved from a database stored on a computer network server, etc.

In certain exemplary embodiments, the invention provides computers (or other apparatus/devices such as biomedical devices or laboratory instrumentation) programmed to carry out the methods described herein. For example, in certain embodiments, the invention provides a computer programmed to receive (i.e., as input) the identity (e.g., the allele(s) or genotype at a location) of one or more genes disclosed herein and provide (i.e., as output) the disease prognosis or other result (e.g., disease diagnosis or prognosis, drug responsiveness, etc.) based on the identity of the genotype at the location. Such output (e.g., communication of disease risk, disease diagnosis or prognosis, drug responsiveness, etc.) may be, for example, in the form of a report on computer readable medium, printed in paper form, and/or displayed on a computer screen or other display.

In various exemplary embodiments, the invention further provides methods of doing business (with respect to methods of doing business, the terms “individual” and “customer” are used herein interchangeably). For example, exemplary methods of doing business can comprise assaying one or more chromosomal locations as disclosed herein and providing a report that includes, for example, a customer's prognosis with respect to CLL and/or that includes the allele(s)/genotype at the assayed locations which may optionally be linked to information (e.g., journal publications, websites, etc.) pertaining to disease risk or other biological/medical significance such as by means of a hyperlink (the report may be provided, for example, on a computer network server or other computer readable medium that is internet-accessible, and the report may be included in a secure database that allows the customer to access their report while preventing other unauthorized individuals from viewing the report), and optionally transmitting the report. Customers (or another party who is associated with the customer, such as the customer's doctor, for example) can request/order (e.g., purchase) the test online via the interne (or by phone, mail order, at an outlet/store, etc.), for example, and a kit can be sent/delivered (or otherwise provided) to the customer (or another party on behalf of the customer, such as the customer's doctor, for example) for collection of a biological sample from the customer (e.g., a blood sample), and the customer (or a party who collects the customer's biological sample) can submit their biological samples for assaying (e.g., to a laboratory or party associated with the laboratory such as a party that accepts the customer samples on behalf of the laboratory, a party for whom the laboratory is under the control of (e.g., the laboratory carries out the assays by request of the party or under a contract with the party, for example), and/or a party that receives at least a portion of the customer's payment for the test). The report may be provided to the customer by, for example, the laboratory that assays the genes or a party associated with the laboratory (e.g., a party that receives at least a portion of the customer's payment for the assay, or a party that requests the laboratory to carry out the assays or that contracts with the laboratory for the assays to be carried out) or a doctor or other medical practitioner who is associated with (e.g., employed by or having a consulting or contracting arrangement with) the laboratory or with a party associated with the laboratory, or the report may be provided to a third party (e.g., a doctor, genetic counselor, hospital, etc.) which optionally provides the report to the customer. In further embodiments, the customer may be a doctor or other medical practitioner, or a hospital, laboratory, medical insurance organization, or other medical organization that requests/orders (e.g., purchases) tests for the purposes of having other individuals (e.g., their patients or customers) assayed for one or more genomic locations disclosed herein and optionally obtaining a report of the assay results.

Those of skill in the art should, in light of the present disclosure, appreciate that many changes can be made in the specific embodiments which are disclosed and still obtain a like or similar result without departing from the spirit and scope of the invention. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Accordingly, the invention is limited only by the following claims.

We claim:

1. A method for determining the prognosis for or predicting responsiveness to a therapeutic regimen for a subject with chronic lymphocytic leukemia (CLL) comprising:
determining the copy number in chromosome 17p and/or 11q or the presence of mutations in p53 and/or ATM in a sample from the subject, wherein a correlation between copy number alterations and the presence of mutations is indicative of a subject's prognosis or likelihood of responsiveness to a therapeutic regimen.

2. The method of claim 1, further comprising detecting copy number alterations in chromosome 11q, trisomy12, 13q and/or 17p.

3. The method of claim 1, further comprising detecting mutations in one or more of p53, ATM, SF3B1, MYD88, FBXW7, NOTCH1, BIRC3, IRF4 and any combination thereof.

4. The method of claim 1, wherein there is a deletion in 13q containing all or a portion of the RB gene.

5. The method of claim 1, wherein there is a deletion in 11q containing all or a portion of BIRC3.

6. The method of claim 1, wherein the distribution of mutations and wild type sequences determines the clonal frequency using next generation sequencing or PCR.

7. The method of claim 1, wherein malignant conditions with elevated WBCs are further evaluated be analyzing mutations in BRAF or NOTCH2, or translocations in chromosomes 11;14 or 14;18.

8. The method of claim 1, wherein alterations of ATM or 11q predict need for alkylator based therapy.

9. The method of claim 1, wherein alterations of p53, 17p, SF3B1, BIRC3 predict lack of benefit from fludarabine or other purine based therapy.

10. The method of claim 1, wherein adverse prognosis is conferred by alterations in p53, 17p, Notch, SF3B1, BIRC3 or a combination thereof.

11. The method of claim 1, wherein the sample is a blood sample, lymph node or bone marrow aspirate.

12. The method of claim 6, wherein B cells are isolated from the sample.

13. A kit for determining the prognosis for or predicting responsiveness to a therapeutic regimen for a subject with chronic lymphocytic leukemia (CLL) comprising: primers specific for amplification of chromosome 11q, 17p or mutations in p53 and ATM.

14. The kit of claim 13, wherein the amplification is performed in a next generation sequencing multiplex format.

15. The kit of claim 13, further comprising primers for amplification of at least one of chromosome 13q, trisomy 12, or mutations in SF3B1, MYD88, FBXW7, NOTCH1, BIRC3, IRF4, and any combination thereof.

16. A method for determining prognosis for a subject having CLL comprising determining the extent of copy number alterations of chromosome 13q, wherein a deletion including the RB gene is indicative of a poor prognosis as compared with a subject not having a deletion including the RB gene.

17. The method of claim 1 or 16, further comprising determining the sequence of the BCR gene.

18. The kit of claim 13, further comprising primers to determine the sequence of the BCR gene.

19. The method of claim 17, wherein the BCR gene is mutated, unmutated and/or stereotyped.
